(19) United States
(12) Patent Application Publication
(10) Pub. No.: US 2020/0181121 A1
(43) Pub. Date: Jun. 11, 2020

(54) Cocrystal of Telmisartan and Hydrochlorothiazide

(71) Applicants: Xuefeng Mei, Shanghai (CN); Jianrong Wang, Shanghai (CN); Qihui Yu, Shanghai (CN)

(72) Inventors: Xuefeng Mei, Shanghai (CN); Jianrong Wang, Shanghai (CN); Qihui Yu, Shanghai (CN)

(73) Assignee: SHANGHAI INSTITUTE OF MATERIA MEDICA, CHINESE ACADEMY OF SCIENCES, Shanghai (CN)

(21) Appl. No.: 16/791,916

(22) Filed: Feb. 14, 2020

Related U.S. Application Data
(63) Continuation of application No. PCT/CN2018/099349, filed on Aug. 8, 2018.

(30) Foreign Application Priority Data
Aug. 15, 2017 (CN) 201710698054.3

(51) Int. Cl. C07D 403/10; C07D 285/20

(52) U.S. Cl. CPC C07D 403/10; C07B 2200/13; C07D 285/20

(57) ABSTRACT

The present disclosure relates to a cocrystal of telmisartan and hydrochlorothiazide, a preparation method and use thereof. In the cocrystal, the molar ratio of telmisartan and hydrochlorothiazide is 1:1. The cocrystal of telmisartan and hydrochlorothiazide was characterized by X-ray powder diffraction (XRPD), proton nuclear magnetic resonance spectra (1H-NMR), thermal gravimetric analysis (TG), scanning differential calorimetry (DSC) and infrared (IR) spectra, and it was found that the maximum plasma concentration of the cocrystal in SD rats was higher than that of any one of hydrochlorothiazide and telmisartan itself. The cocrystal of telmisartan and hydrochlorothiazide has a simple preparation method and good physical and chemical properties.

### CROSS-REFERENCE TO RELATED PATENT APPLICATIONS

This patent application is a continuation of PCT/CN2018/099349, filed Aug. 8, 2018, which claims priority to Chinese Application No. 201710698054.3, filed Aug. 15, 2017, the entire teachings and disclosure of which are incorporated herein by reference thereto.

### FIELD OF THE INVENTION

The present invention relates to the field of pharmaceutical chemistry and crystallization process, specifically, to the cocrystal of telmisartan and hydrochlorothiazide, the preparation method and use thereof.

### BACKGROUND OF THE INVENTION

The Chemical name of Telmisartan is 4′-[[2-propyl-4-methyl-6(1-methyl-benzimidazol-2-yl)-benzimidazol-1-yl]methyl]-2-biphenylcarboxylic acid, and its chemical structure is shown as follows:

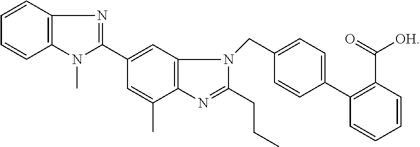 [SMILES: CCCc1nc2c(C)cc(-c3nc4ccccc4n3C)cc2n1Cc1ccc(-c2ccccc2C(=O)O)cc1]

Telmisartan is a specific angiotensin II receptor antagonists, and telmisartan has the strongest affinity to angiotensin II receptor type 1 (AT1 receptor) and thus has a high selective binding. Angiotensin II is a polypeptide-based compound composed of eight amino acid residues and mainly converted from angiotensin I by angiotensin-converting enzyme, and it has main physiological functions of contracting blood vessels, promoting the secretion of norepinephrine and aldosterone and promoting the reabsorption of Na⁺. Thus, telmisartan can treat hypertension by affecting the renin-angiotensin-aldosterone system (RAAS system) in vivo. More importantly, telmisartan molecules may combine with the receptor site of the peroxisome proliferator-activated receptor γ (PPARγ) molecule in the form of a ligand, excite PPARγ, and regulate the glucose and lipid metabolism in vivo.

The chemical name of hydrochlorothiazide is 6-chloro-3,4-dihydro-2H-1,2,4-benzothiadiazine-7-sulfonamido-1,1-dioxide, and its chemical structure is as follows:

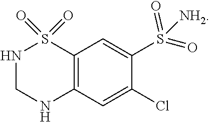 [SMILES: NS(=O)(=O)c1cc2c(cc1Cl)NCNS2(=O)=O]

Hydrochlorothiazide is a thiazide-type moderate efficacy diuretics, which works by inhibiting Na⁺—Cl⁻ co-transport in distal convoluted tubule, reducing the reabsorption of Na⁺ in the original urine, thereby increasing the exchange of Na⁺—K⁺ in distal tubule and collecting tubes, and increasing the secretion of K⁺. This kind of medicine can inhibit phosphodiesterase activity, reduce the renal tubular uptake of fatty acids and mitochondrial oxygen consumption, thereby inhibit the active reabsorption of Na⁺, Cl⁻ in renal tubular. Thus the diuretic effect is realized and the object of treating hypertension can be achieved. The oral absorption of hydrochlorothiazide is rapid, but its metabolism was not complete. It will cause disturbances in the concentration of water and electrolytes in the body, causing side effects such as hypokalemia and hyperuricemia when used for a long time.

It has been found in the research on hypertension that, hypertension is a multifactorial disease and its pathogenesis is very complicated. The effective rate of single drug treatment is only 50% to 60% even for mild hypertension. Although increasing the dose can improve the efficacy, it will also increase the adverse reactions and even result in a drug resistance. A large number of clinical practices have shown that the co-administration of medication has obvious advantages. The combined administration of small doses of antihypertensive drugs with different mechanisms can exert the synergistic effect of different drugs. Among them, the combined use of thiazide diuretics with angiotensin-converting enzyme inhibitors (ACE inhibitors), angiotensin receptor antagonists (ARBs), calcium channel blockers is clinically preferred. Thiazide diuretics can reduce plasma volume and thus reduce blood pressure, but a decrease in plasma volume will activate the RAAS system, and the resulting vasoconstriction and increased aldosterone secretion will partially offset the antihypertensive effect of diuretics. ARB inhibits the RAAS system, thereby a synergistic effect with diuretics can be achieved in terms of reducing blood pressure. This medication plan combines the effects of the drugs, resulting in an increase in the efficacy of many patients, and the combined administration of ARBs and thiazide diuretics reduces the side effects of thiazide diuretics alone and improves safety. In addition, this combined administration can increase compliance and is more acceptable for patients. The compound drug of telmisartan and hydrochlorothiazide has been widely used since being listed in 1999.

The present inventors designed and synthesized a new cocrystal of telmisartan and hydrochlorothiazide. The maximum plasma concentration and the area under the curve (AUC) of the cocrystal in SD rats in vivo are significantly improved than the hydrochlorothiazide or telmisartan alone or the conventional physical mixture thereof. Thus a new practical means can be provided to realize the bioavailability of telmisartan and hydrochlorothiazide, as well as the clinical efficacy thereof.

### SUMMARY OF THE INVENTION

It is the first object of the present invention to provide a cocrystal of telmisartan and hydrochlorothiazide.

It is the second object of the present invention to provide a preparation method of the cocrystal of telmisartan and hydrochlorothiazide.

It is the third object of the present invention to provide a pharmaceutical composition, said pharmaceutical composition comprises the above-described cocrystal of telmisartan and hydrochlorothiazide, and a pharmaceutically acceptable carrier.

It is the fourth object of the present invention to provide a use of the cocrystal of telmisartan and hydrochlorothiazide in preparation of a drug for the treatment of cardiovascular and cerebrovascular diseases, such as hypertension, congestive heart failure, etc.

According to the first aspect of the present invention, provided is a cocrystal of telmisartan and hydrochlorothiazide, in which the molar ratio of telmisartan and hydrochlorothiaziden is 1:1.

In the X-ray powder diffraction pattern of the cocrystal of telmisartan and hydrochlorothiazide, characteristic peaks exist at the 2θ angle of about 5.56°±0.2°, 14.68°±0.2°, 15.47°±0.2°.

Preferably, in the X-ray powder diffraction pattern of the cocrystal of telmisartan and hydrochlorothiazide, characteristic peaks exist at the 2θ angle of about 5.56°±0.2°, 9.96°±0.2°, 11.13°±0.2°, 14.68°±0.2°, 15.47°±0.2°, 17.72°±0.2°, 18.35°±0.2°, 19.43°±0.2°.

Further preferably, in the X-ray powder diffraction pattern of the cocrystal of telmisartan and hydrochlorothiazide, characteristic peaks exist at the 2θ angle of about 5.56°±0.2°, 7.41°±0.2°, 9.96°±0.2°, 11.13°±0.2°, 12.30°±0.2°, 14.68°±0.2°, 15.47°±0.2°, 17.72°±0.2°, 18.35°±0.2°, 19.43°±0.2°, 21.20°±0.2°, 22.15°±0.2°, 24.30°±0.2°, 24.81°±0.2°.

In particular, in the X-ray powder diffraction pattern of the cocrystal of telmisartan and hydrochlorothiazide, the pattern is shown substantially as the XRPD pattern of FIG. 1.

Due to different measurement conditions, the 2θ angle and relative intensity of each peak in the XRPD pattern may vary. The 2θ angle change is generally within ±0.2°, but it may also exceed this range slightly. Those skilled in the art will understand that the relative intensity may depend, for example, on the sample formulation or the used equipment.

The cocrystal of telmisartan and hydrochlorothiazide is characterized in that, a characteristic melting peak at about 199.02±0.2° C. (onset temperature) exists in the differential scanning calorimetry spectrum of the cocrystal of telmisartan and hydrochlorothiazide. The cocrystal of telmisartan and hydrochlorothiazide has a differential scanning calorimetry (DSC) spectrum as substantially shown in FIG. 4.

In the infrared spectrum of the cocrystal of telmisartan and hydrochlorothiazide, characteristic peaks exist at least at about 3378 cm⁻¹, 3271 cm⁻¹, 3168 cm⁻¹, 3048 cm⁻¹, 2965 cm⁻¹, 2932 cm⁻¹, 2872 cm⁻¹, 2649 cm⁻¹, 1920 cm⁻¹, 1594 cm⁻¹, 1506 cm⁻¹, 1455 cm⁻¹, 1381 cm⁻¹, 1353 cm⁻¹, 1316 cm⁻¹, 1274 cm⁻¹, 1167 cm⁻¹, 1083 cm⁻¹, 1051 cm⁻¹, 1032 cm⁻¹, 1009 cm⁻¹, 861 cm⁻¹, 809 cm⁻¹, 754 cm⁻¹, 712 cm⁻¹, 670 cm⁻¹, 609 cm⁻¹, 549 cm⁻¹.

According to the second aspect of the present invention, a preparation method of the cocrystal of telmisartan and hydrochlorothiazide is provided, and the method is one of the following methods:

Method I:

The method I comprises the following steps:

(a) dissolving the hydrochlorothiazide in an organic solvent to prepare a saturated solution of hydrochlorothiazide;

(b) dissolving separately the telmisartan in the same organic solvent as that in step (a) to prepare a saturated solution of telmisartan;

(c) mixing and stirring the saturated solution of hydrochlorothiazide in the step (a) and the saturated solution of telmisartan in the step (b), thereby forming a cocrystal of telmisartan and hydrochlorothiazide;

(d) separating the cocrystal of telmisartan and hydrochlorothiazide formed in the step (c), so as to obtain the cocrystal of telmisartan and hydrochlorothiazide;

Method II:

The method II comprises the following steps:

(e) dissolving the hydrochlorothiazide in an organic solvent to prepare a saturated solution of hydrochlorothiazide;

(f) adding a telmisartan powder to the saturated solution of hydrochlorothiazide, suspending until forming a supersaturated condition to precipitate crystals, thereby forming a cocrystal of telmisartan and hydrochlorothiazide;

(g) separating the cocrystal of telmisartan and hydrochlorothiazide formed in the step (f), so as to obtain the cocrystal of telmisartan and hydrochlorothiazide;

Method III:

The method III comprises the following steps:

(h) dissolving the telmisartan in an organic solvent to prepare a saturated solution of telmisartan;

(i) adding a hydrochlorothiazide powder to the saturated solution of telmisartan, suspending until forming a supersaturated condition to precipitate crystals, thereby forming a cocrystal of telmisartan and hydrochlorothiazide;

(j) separating the cocrystal of telmisartan and hydrochlorothiazide formed in the step (i), so as to obtain the cocrystal of telmisartan and hydrochlorothiazide;

Preferably,

The organic solvent is one or more selected from the group consisting of methyl isobutyl ketone, methanol, ethyl acetate, nitromethane, ethanol, and isopropyl acetate, preferably methanol;

In step (c),

The saturated solution of hydrochlorothiazide and the saturated solution of telmisartan are mixed in a volume ratio of the solution of 1.2:1 to 1:1.2, preferably 1:1;

In step (d), step (g) and step (j),

The separating includes:

(d1) obtaining the cocrystal of telmisartan and hydrochlorothiazide by filtration; or

(d2) obtaining the cocrystal of telmisartan and hydrochlorothiazide by centrifugation and filtration; or

(d3) after separating the cocrystal of telmisartan and hydrochlorothiazide in the step (d1) or (d2), further evaporating to remove the liquid solution separated in the step (d1) or (d2), so as to obtain the cocrystal of telmisartan and hydrochlorothiazide.

In steps (f) and (i),

The molar ratio of telmisartan and hydrochlorothiazide can fluctuate within a certain range, and it has no effect on the quality of the cocrystal, the range is 1.2:1 to 1:1.2, preferably 1.1:1 to 1:1.1, more preferably 1.05:1 to 1:1.05, and most preferably 1:1.

The third aspect of the present invention relates to a pharmaceutical composition, said pharmaceutical composition comprises the cocrystal of telmisartan and hydrochlorothiazide, and a pharmaceutically acceptable carrier.

The fourth aspect of the present invention relates to a use of the cocrystal of telmisartan and hydrochlorothiazide and/or the pharmaceutical composition described above in preparation of a drug for the treatment of cardiovascular and cerebrovascular diseases, such as hypertension, congestive heart failure, etc.

### Technical Effect

The present invention provides a cocrystal of telmisartan and hydrochlorothiazide. The preparation method thereof is simple, reproducible, and it is easy to control the process of forming the cocrystal. Compared with hydrochlorothiazide or telmisartan itself, the maximum plasma concentration of cocrystal in SD rats was higher than that of hydrochlorothiazide or telmisartan. It can be seen from the pharmacokinetic parameters of hydrochlorothiazide that, the maximum concentration of the cocrystal sample is 1.24 times that of the separate administration group, and the area under the curve of the cocrystal sample is 1.65 times that of the separate administration group. It can be seen from the pharmacokinetic parameters of telmisartan that, the maximum concentration of the cocrystal sample is 5.64 times that of the separate administration group, and the area under the curve of the cocrystal sample is 5.0 times that of the separate administration group. Thus it can be seen that the cocrystal samples have improved the pharmacokinetic behavior of hydrochlorothiazide and telmisartan, especially, the cocrystal samples can significantly increase the content of telmisartan exposed in vivo. In addition, the co-administration of the two drugs has a great effect on the treatment of hypertension, and can achieve clinical effects that cannot be achieved with any one of the two samples solely.

### BRIEF DESCRIPTIONS OF THE DRAWINGS

FIG. 1 is the X-ray powder diffraction (XRPD) pattern of the cocrystal of telmisartan and hydrochlorothiazide of the Example 1 of the present invention;

FIG. 2 is the one-dimensional proton nuclear magnetic resonance spectra (¹H NMR) of the cocrystal of telmisartan and hydrochlorothiazide of the Example 1 of the present invention;

FIG. 3 is the thermal gravimetric analysis (TG) curve of the cocrystal of telmisartan and hydrochlorothiazide of the Example 1 of the present invention;

FIG. 4 is the differential scanning calorimetry (DSC) diagram of the cocrystal of telmisartan and hydrochlorothiazide of the Example 1 of the present invention;

FIG. 5 is the infrared spectrum (IR) of the cocrystal of telmisartan and hydrochlorothiazide of the Example 1 of the present invention;

FIG. 6 is the hydrochlorothiazide concentration-time curve in SD rat in vivo of the cocrystal of telmisartan and hydrochlorothiazide of the Example 1 of the present invention;

FIG. 7 is the telmisartan concentration-time curve in SD rat in vivo of the cocrystal of telmisartan and hydrochlorothiazide of the Example 1 of the present invention.

### DETAILED DESCRIPTION

Hereinafter, the present invention is further illustrated with reference to specific embodiments, but the present invention is not limited thereto.

The testing instruments and methods are as follows:

The instrument used for X-ray powder diffraction (XRPD) was Bruker D8 Advance diffractometer, using Kα ray (line with K1.5418 Å) of Cu, with the voltage of 40 kV and current of 40 mA. The instrument is corrected for the peak position using the standard sample that comes with the instrument before using. The acquisition software is Diffrac Plus XRD Commander and the analysis software is MDI Jade 6.0. The samples were tested at room temperature, and the samples to be detected are put on the organic glass slide. The detailed test conditions are as follows: 2θ angle range: 3 to 40°, step size: 0.02°; speed: 0.1 second/step. Unless otherwise specified, the samples were not ground before testing.

The data of proton nuclear magnetic resonance spectra (¹H NMR) was collected using Mercury-Plus 400 from Varian company of USA. The sample was prepared using deuterated DMSO solution, and the solvent peak thereof was at 2.50 ppm. The analysis software is MestReNova.

The thermogravimetric analysis (TGA) data was collected using TG20F3 from NETZSCH Scientific Instrument Co., Ltd. of Germany, the control software of the instrument was NETZSCH-Proteus-6, and the analysis software was Proteus Analysis. The sample was heated from room temperature to 400° C. at a heating rate of 10° C./min under the protection of 50 mL/min of dry nitrogen, and the weight change of the sample during the heating process was recorded by the software in the same time.

The data of differential thermal analysis (DSC) was collected using DSC Q2000 differential scanning calorimeter of TA Instrument Company of USA. The control software of the instrument was Thermal Advantage, and the analysis software was Universal Analysis. The sample was heated from room temperature to 200° C. at a heating rate of 10° C./min under the protection of 50 mL/min of dry nitrogen, and the heat change of the sample during the heating process was recorded by the TA software in the same time.

The infrared analysis (IR) was performed at room temperature using the Nicolet-Magna FT-IR 750 infrared spectrometer from Nichols, USA. The detection wave number range is 4000-350 cm⁻¹.

The reagents such as methanol are of analytical grade and are provided by Sinopharm Chemical Reagent Co., Ltd. The reagents and solvents used are specially treated unless otherwise specified. The active pharmaceutical ingredients (APIs) of hydrochlorothiazide and telmisartan were purchased from Adamas Reagent Company, with a purity of more than 99%. All temperatures are presented in ° C. (degrees Centigrade), and the room temperature means 20 to 25° C.

### EXAMPLE 1

Cocrystal of Telmisartan and Hydrochlorothiazide

At room temperature, a saturated solution of hydrochlorothiazide (29.7 g) was formed in 200 mL of methanol solution, and the supernatant was taken by filtration. Similarly, a saturated solution of telmisartan (51.5 g) was formed in 200 mL of methanol solution, and the supernatant was taken by filtration. The saturated solution of hydrochlorothiazide and the saturated solution of telmisartan were added to the beaker in an equal volume ratio, and were suspended until forming a supersaturated state. The resultant was centrifugated and filtrated, so as to obtain the cocrystal of telmisartan and hydrochlorothiazide (75.6 g).

The produced cocrystal of telmisartan and hydrochlorothiazide was characterized by X-ray powder diffraction (XRPD), proton nuclear magnetic resonance spectra (¹H-NMR), thermal gravimetric analysis (TG), differential scanning calorimetry (DSC) and infrared (IR) spectra.

The analysis result of proton nuclear magnetic resonance spectra (¹H-NMR) is shown in the FIG. 2, the result of the proton nuclear magnetic resonance spectra showed that the molar ratio of telmisartan and hydrochlorothiazide is 1:1. The two hydrogen atoms in the −CH₂— segment in the structure of hydrochlorothiazide molecular have a chemical shift of 4.7 ppm, and the two hydrogen atoms in the —CH₂— segment in the structure of telmisartan molecular have a chemical shift of 5.7 ppm. In the ¹H-NMR spectrum of the cocrystal, it can be seen that there are peaks at both chemical shifts, and the ratio of the integrated area of them is 1:1. Therefore, it can be determined that the molar ratio of two molecules in the cocrystal is 1:1.

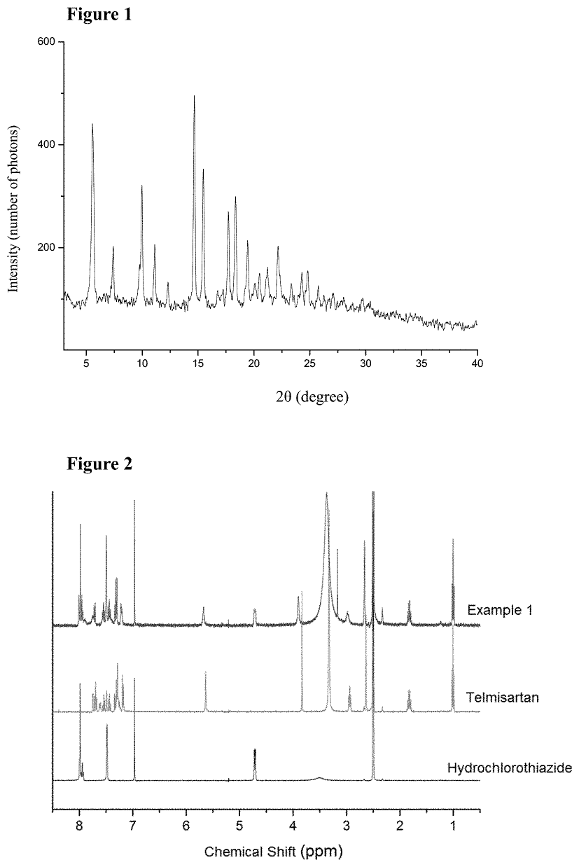

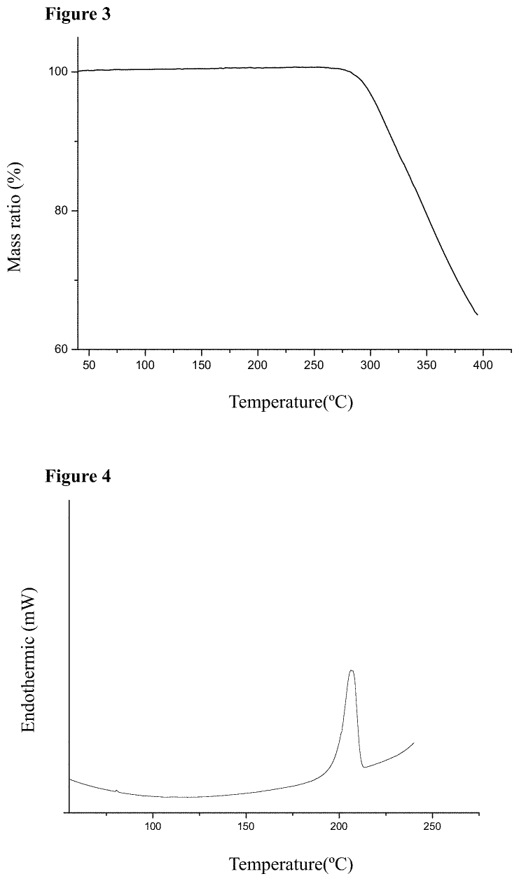

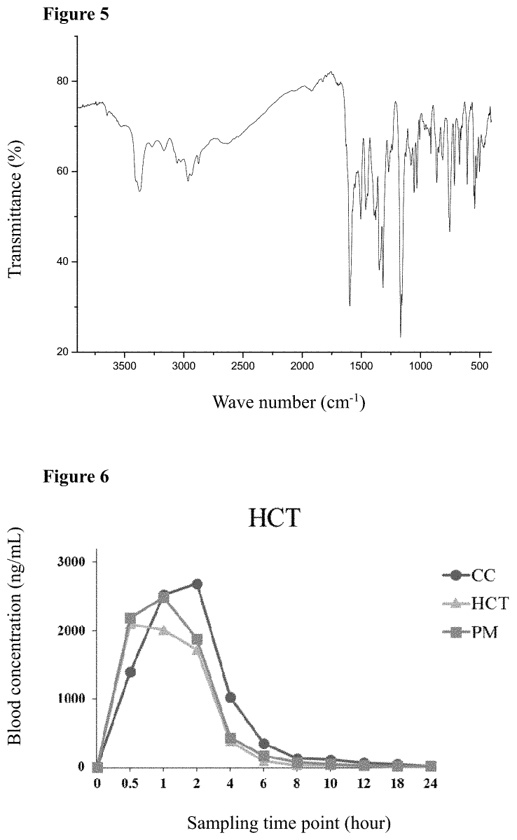

The results of X-ray powder diffraction analysis are shown in FIG. 1; the results of thermogravimetric analysis are shown in FIG. 3; the results of differential scanning calorimetry analysis are shown in FIG. 4; and the results of infrared analysis are shown in FIG. 5.

### EXAMPLE 2

Cocrystal of Telmisartan and Hydrochlorothiazide

At room temperature, hydrochlorothiazide (35.6 g) was dissolved in a methanol solution (200 mL) to form a saturated solution, and the supernatant was taken by filtration. A telmisartan powder (61.8 g) was added into the saturated solution of hydrochlorothiazide in methanol, the resultant was suspended until forming a supersaturated condition to precipitate crystals, so as to form cocrystal of hydrochlorothiazide and telmisartan. The resultant was centrifugated and filtrated, so as to obtain the cocrystal of hydrochlorothiazide and telmisartan (87.4 g).

### EXAMPLE 3

Cocrystal of Hydrochlorothiazide and Telmisartan

At room temperature, telmisartan (66.9 g) was dissolved in a methanol solution (250 mL) to form a saturated solution, and the supernatant was taken by filtration. A hydrochlorothiazide powder (46.2 g) was added into the saturated solution of telmisartan in methanol, the resultant was suspended until forming a supersaturated condition to precipitate crystals, so as to form cocrystal of hydrochlorothiazide and telmisartan. The resultant was centrifugated and filtrated, so as to obtain the cocrystal of hydrochlorothiazide and telmisartan (100.2 g).

The produced cocrystal of hydrochlorothiazide and telmisartan in the Examples 2 and 3 were characterized by the solid chemical characterization method, such as X-ray powder diffraction (XRPD), proton nuclear magnetic resonance spectra (¹H-NMR), thermal gravimetric analysis (TG), differential scanning calorimetry (DSC) and infrared (IR) spectra. The results thereof are substantially the same as those of the cocrystal of hydrochlorothiazide and telmisartan prepared in the Example 1.

### EXPERIMENTAL EXAMPLE 1

Comparison of the maximum plasma concentration of cocrystal of hydrochlorothiazide and telmisartan in SD rats with that of hydrochlorothiazide or telmisartan itself.

The source of the test sample: cocrystal of hydrochlorothiazide and telmisartan prepared in Example 1, and hydrochlorothiazide and telmisartan APIs purchased from Adamas Reagent Company.

The experimental method is as follows:

The rats (females) were randomly divided into 4 groups with 6 rats per each group. The rats were fasted for 12 h but can drink freely before the test, and then were weighed.

Mode of Administration: Administrate with Suspension Agent

Administration dose: calculated according to the rat mass of 200 g for each one, formulated in a 0.5% sodium carboxymethylcellulose CMCNa aqueous solution.

| | | Suspension | | Concen- |
|---|---|---|---|---|
| | Administration | medium dose | Administration | tration |
| | dose (mg/kg) | (mL) | mass (mg/rat) | (mg/mL) |
| HCT | 2.3 | 2 | 0.46 | 0.23 |
| TEL | 4 | 2 | 0.8 | 0.4 |
| Cocrystal | 6.3 | 2 | 1.26 | 0.63 |
| Physical | HCT 2.3 and | 2 | 1.26 | 0.63 |
| mixture | TEL 4 | | | |

The 4 groups were administrated intragastrically with HCT (hydrochlorothiazide), TEL (telmisartan), CC (co-crystal of telmisartan and hydrochlorothiazide) and PM (physical mixture of hydrochlorothiazide and telmisartan) respectively (approximately 2 mL). The rats were fed 3 h after the drug-administration. The 4 groups were administrated intragastrically with HCT, TEL, CC and PM respectively (approximately 2 mL). The rats were fed 3 h after the drug-administration. The blood collection site was orbital blood collection, blood collection time points: 0, 30 min, 1 h, 2 h, 4 h, 6 h, 8 h, 10 h, 12 h, 18 h, 24 h. 200 μL of blood (heparinized tube) were taken, which was centrifuged at 10,000 rpm for 5 min (at 10° C.). The plasma was separated, freezed at −20° C. in refrigerator, and used for blood concentration test after freezing.

Biological Sample Test Method:

Instrument: Shimadzu LC-MS 8030

MS Conditions:

| | | | Internal |
|---|---|---|---|
| | Hydrochlo- | | standard |
| MS conditions | rothiazide | Telmisartan | (tolbutamide) |
| Ion pair detected in Q1 | 296/268.9 | 513.3/469.25 | 269.1/169.85 |
| chamber/Ion pair detected | | | |
| in Q3 chamber | | | |
| Collision energy (eV) | 18 | 22 | 15 |

| | |
|---|---|
| Interface temperature (° C.) | 400 |
| DL pipe temperature (° C.) | 250 |
| Spraying gas flow | 3 |
| (mL/min) | |
| Heater component | 400 |
| temperature (° C.) | |
| Dry gas flow (mL/min) | 15 |

### Chromatographic Conditions:

| Packed column | shim-pack GVP-ODS 5L * 2.0 (SHIMADZU) | | | |
|---|---|---|---|---|
| Chromatographic | ACE column | | | |
| column | | | | |
| Column temperature | 40° C. | | | |
| Mobile phase A | Acetonitrile | | | |
| Mobile phase C | Aqueous solution containing 0.2% formic acid | | | |
| | and 5 mmol of ammonium acetate | | | |
| Autosampler | MeOH—H₂O 1:1 | | | |
| cleaning solution | | | | |
| | | Flow | | |
| | Time | rate | A | C |
| Elution gradient | (min) | (mL/min) | (%) | (%) |
| | 0.01 | 0.2 | 60 | 40 |
| | 5 | 0.2 | STOP | |

| Total detection time | 5 min | |
|---|---|---|
| Injection amount | 10 μL | |
| Each retention time | HCT, TEL, IS | 1.5 min, 2.3 min, |
| | | 2.4 min |

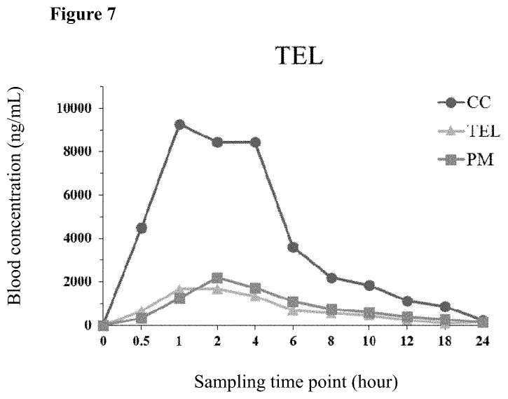

The results are shown in FIG. 6 and FIG. 7. It can be shown that under the condition that the hydrochlorothiazide, telmisartan and cocrystal thereof are administered with the same dose by intragastric administration, both the area under the curve (AUC) and the maximum concentration (Cmax) in the drug-time curve of the cocrystal are much higher than those of hydrochlorothiazide and telmisartan. It can be seen from the pharmacokinetic parameters of HCT that, the maximum concentration of the cocrystal sample is 1.24 times that of the separate administration group, and the AUC of the cocrystal sample is 1.65 times that of the separate administration group. It can be seen from the pharmacokinetic parameters of TEL that, the maximum concentration of the cocrystal sample is 5.64 times that of the separate administration group, and the AUC of the cocrystal sample is 5.0 times that of the separate administration group. Further, the AUC of cocrystal sample is also higher than the AUC of the normal mixed samples with the same dose. In particular, for telmisartan, the AUC of the cocrystal sample is considerably higher than the AUC of the normal mixed samples with the same dose. Thus it can be seen that the cocrystal samples have improved pharmacokinetic behavior of hydrochlorothiazide and telmisartan, especially, the cocrystal samples can significantly increase the content of telmisartan exposed in vivo.

### EXPERIMENTAL EXAMPLE 2

According to the description in “Stability Data Evaluation” of ICH guidelines Q1E, a comparison of accelerated stability test (at 40° C.±2° C., RH 75±5%) of cocrystal of hydrochlorothiazide and telmisartan obtained in Example 1 with that of telmisartan or hydrochlorothiazide itself was conducted respectively. The cocrystal of telmisartan and hydrochlorothiazide, telmisartan, and hydrochlorothiazide were double packaged using the polyethylene-sealed bag respectively, stood for 1, 2 or 3 months under the accelerated stability test conditions (at 40° C.±2° C., RH 75±5%). Then the samples were taken and measured for the change of the content.

The Results of the Content Test in the Accelerated Test

| | | Hydrochlo- | Telmisartan | Hydrochlo- |
|---|---|---|---|---|
| time | Telmisartan | rothiazide | in cocrystal | rothiazide in |
| (month) | (%) | (%) | (%) | cocrystal (%) |
| 0 | 100 | 100 | 100 | 100 |
| 1 | 97.28 | 97.37 | 99.46 | 100.19 |
| 2 | 94.47 | 96.34 | 100.15 | 100.75 |
| 3 | 94.20 | 95.28 | 99.13 | 99.77 |

As can be seen from the results of accelerated tests, after 3 months, the contents of telmisartan and hydrochlorothiazide in the cocrystal were substantially maintained as the initial contents, but the telmisartan alone degraded to 94.20%, and hydrochlorothiazide alone degraded to 95.28%. It can be seen that, compared to the telmisartan and hydrochlorothiazide, the cocrystal of telmisartan and hydrochlorothiazide has better stability in the accelerated test, thus can effectively extend the shelf life of the drug.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A cocrystal of telmisartan and hydrochlorothiazide, characterized in that the molar ratio of telmisartan and hydrochlorothiazide in the cocrystal is 1:1.

2. The cocrystal of telmisartan and hydrochlorothiazide according to claim 1, characterized in that, in the X-ray powder diffraction pattern of the cocrystal of telmisartan and hydrochlorothiazide, characteristic peaks exist at the 2θ angle of about 5.56°±0.2°, 9.96°±0.2°, 11.13°±0.2°, 14.68°±0.2°, 15.47°±0.2°, 17.72°±0.2°, 18.35°±0.2°, 19.43°±0.2°.

3. The cocrystal of telmisartan and hydrochlorothiazide according to claim 1, characterized in that, in the X-ray powder diffraction pattern of the cocrystal of telmisartan and hydrochlorothiazide, characteristic peaks exist at the 2θ angle of about 5.56°±0.2°, 7.41°±0.2°, 9.96°±0.2°, 11.13°±0.2°, 12.30°±0.2°, 14.68°±0.2°, 15.47°±0.2°, 17.72°±0.2°, 18.35°±0.2°, 19.43°±0.2°, 21.20°±0.2°, 22.15°±0.2°, 24.30°±0.2°, 24.81°±0.2°.

4. The cocrystal of telmisartan and hydrochlorothiazide according to claim 1, characterized in that, the X-ray powder diffraction pattern of the cocrystal of telmisartan and hydrochlorothiazide is shown substantially as the XRPD pattern of FIG. 1.

5. The cocrystal of telmisartan and hydrochlorothiazide according to claim 1, characterized in that, a characteristic melting peak at about 199.02±0.2° C. exists in the differential scanning calorimetry spectrum of the cocrystal of telmisartan and hydrochlorothiazide.

6. A preparation method for the cocrystal of telmisartan and hydrochlorothiazide of claim 1, the method is one of the following methods:
Method I:
The method I comprises the following steps:
(a) dissolving the hydrochlorothiazide in an organic solvent to prepare a saturated solution of hydrochlorothiazide;
(b) dissolving separately the telmisartan in the same organic solvent as that in step (a) to prepare a saturated solution of telmisartan;
(c) mixing and stirring the saturated solution of hydrochlorothiazide in the step (a) and the saturated solution of telmisartan in the step (b), thereby forming the cocrystal of telmisartan and hydrochlorothiazide;
(d) separating the cocrystal of telmisartan and hydrochlorothiazide formed in the step (c), so as to obtain the cocrystal of telmisartan and hydrochlorothiazide;
Method II:
The method II comprises the following steps:
(e) dissolving the hydrochlorothiazide in an organic solvent to prepare a saturated solution of hydrochlorothiazide;
(f) adding a telmisartan powder to the saturated solution of hydrochlorothiazide, suspending until forming a supersaturated condition to precipitate crystals, thereby forming the cocrystal of telmisartan and hydrochlorothiazide;
(g) separating the cocrystal of telmisartan and hydrochlorothiazide formed in the step (f), so as to obtain the cocrystal of telmisartan and hydrochlorothiazide;
Method III:
The method III comprises the following steps:
(h) dissolving the telmisartan in an organic solvent to prepare a saturated solution of telmisartan;
(i) adding a hydrochlorothiazide powder to the saturated solution of telmisartan, suspending until forming a supersaturated condition to precipitate crystals, thereby forming the cocrystal of telmisartan and hydrochlorothiazide;
(j) separating the cocrystal of telmisartan and hydrochlorothiazide formed in the step (i), so as to obtain the cocrystal of telmisartan and hydrochlorothiazide.

7. The preparation method for the cocrystal of telmisartan and hydrochlorothiazide according to claim 6, characterized in that,
the organic solvent is one or more selected from the group consisting of methanol, ethanol, isopropanol, n-propanol, isopentanol, acetonitrile, methyl ethyl ketone, ethyl acetate, methyl isobutyl ketone, preferably methanol;
In step (d), step (g) and step (j), the separating comprises:
(d1) obtaining the cocrystal of telmisartan and hydrochlorothiazide by filtration; or
(d2) obtaining the cocrystal of telmisartan and hydrochlorothiazide by centrifugation and filtration; or
(d3) after separating the cocrystal of telmisartan and hydrochlorothiazide in the step (d1) or (d2), further evaporating to remove the liquid solution separated in the step (d1) or (d2), so as to obtain the cocrystal of telmisartan and hydrochlorothiazide.

8. A pharmaceutical composition, said pharmaceutical composition comprises the cocrystal of telmisartan and hydrochlorothiazide of claim 1, and a pharmaceutically acceptable carrier.

9. Use of the cocrystal of telmisartan and hydrochlorothiazide of claim 1 or a pharmaceutical composition comprising the cocrystal of telmisartan and hydrochlorothiazide of claim 1 and a pharmaceutically acceptable carrier in preparation of a drug for the treatment of cardiovascular and cerebrovascular diseases.

10. A cocrystal of telmisartan and hydrochlorothiazide, characterized in that, in the X-ray powder diffraction pattern of the cocrystal of telmisartan and hydrochlorothiazide, characteristic peaks exist at the 2θ angle of about 5.56°±0.2°, 14.68°±0.2°, 15.47°±0.2°.

11. The cocrystal of telmisartan and hydrochlorothiazide according to claim 10, characterized in that, in the X-ray powder diffraction pattern of the cocrystal of telmisartan and hydrochlorothiazide, further characteristic peaks exist at the 2θ angle of about 9.96°±0.2°, 11.13°±0.2°, 17.72°±0.2°, 18.35°±0.2°, 19.43°±0.2°.

12. The cocrystal of telmisartan and hydrochlorothiazide according to claim 10, characterized in that, in the X-ray powder diffraction pattern of the cocrystal of telmisartan and hydrochlorothiazide, further characteristic peaks exist at the 2θ angle of about 7.41°±0.2°, 9.96°±0.2°, 11.13°±0.2°, 12.30°±0.2°, 17.72°±0.2°, 18.35°±0.2°, 19.43°±0.2°, 21.20°±0.2°, 22.15°±0.2°, 24.30°±0.2°, 24.81°±0.2°.

13. The cocrystal of telmisartan and hydrochlorothiazide according to claim 10, characterized in that, the X-ray powder diffraction pattern of the cocrystal of telmisartan and hydrochlorothiazide is shown substantially as the XRPD pattern of FIG. 1.

14. The cocrystal of telmisartan and hydrochlorothiazide according to claim 10, characterized in that, a characteristic melting peak at about 199.02±0.2° C. exists in the differential scanning calorimetry spectrum of the cocrystal of telmisartan and hydrochlorothiazide.

15. A preparation method for the cocrystal of telmisartan and hydrochlorothiazide of claim 10, the method is one of the following methods:
Method I:
The method I comprises the following steps:
(a) dissolving the hydrochlorothiazide in an organic solvent to prepare a saturated solution of hydrochlorothiazide;
(b) dissolving separately the telmisartan in the same organic solvent as that in step (a) to prepare a saturated solution of telmisartan;
(c) mixing and stirring the saturated solution of hydrochlorothiazide in the step (a) and the saturated solution of telmisartan in the step (b), thereby forming the cocrystal of telmisartan and hydrochlorothiazide;
(d) separating the cocrystal of telmisartan and hydrochlorothiazide formed in the step (c), so as to obtain the cocrystal of telmisartan and hydrochlorothiazide;
Method II:
The method II comprises the following steps:
(e) dissolving the hydrochlorothiazide in an organic solvent to prepare a saturated solution of hydrochlorothiazide;
(f) adding a telmisartan powder to the saturated solution of hydrochlorothiazide, suspending until forming a supersaturated condition to precipitate crystals, thereby forming the cocrystal of telmisartan and hydrochlorothiazide;
(g) separating the cocrystal of telmisartan and hydrochlorothiazide formed in the step (f), so as to obtain the cocrystal of telmisartan and hydrochlorothiazide;
Method III:
The method III comprises the following steps:
(h) dissolving the telmisartan in an organic solvent to prepare a saturated solution of telmisartan;
(i) adding a hydrochlorothiazide powder to the saturated solution of telmisartan, suspending until forming a supersaturated condition to precipitate crystals, thereby forming the cocrystal of telmisartan and hydrochlorothiazide;
(j) separating the cocrystal of telmisartan and hydrochlorothiazide formed in the step (i), so as to obtain the cocrystal of telmisartan and hydrochlorothiazide.

16. The preparation method for the cocrystal of telmisartan and hydrochlorothiazide according to claim 15, characterized in that,
the organic solvent is one or more selected from the group consisting of methanol, ethanol, isopropanol, n-propanol, isopentanol, acetonitrile, methyl ethyl ketone, ethyl acetate, methyl isobutyl ketone, preferably methanol;
In step (d), step (g) and step (j), the separating comprises:
(d1) obtaining the cocrystal of telmisartan and hydrochlorothiazide by filtration; or
(d2) obtaining the cocrystal of telmisartan and hydrochlorothiazide by centrifugation and filtration; or
(d3) after separating the cocrystal of telmisartan and hydrochlorothiazide in the step (d1) or (d2), further evaporating to remove the liquid solution separated in the step (d1) or (d2), so as to obtain the cocrystal of telmisartan and hydrochlorothiazide.

17. A pharmaceutical composition, said pharmaceutical composition comprises the cocrystal of telmisartan and hydrochlorothiazide of claim 10, and a pharmaceutically acceptable carrier.

18. Use of the cocrystal of telmisartan and hydrochlorothiazide of claim 10 or a pharmaceutical composition comprising the cocrystal of telmisartan and hydrochlorothiazide of claim 10 and a pharmaceutically acceptable carrier in preparation of a drug for the treatment of cardiovascular and cerebrovascular diseases.
